(19) United States
(12) Patent Application Publication
(10) Pub. No.: US 2024/0350575 A1
(43) Pub. Date: Oct. 24, 2024

(54) PLANT EXTRACT FOR ALLEVATING PULMONARY FIBROSIS, AND PREPARATION METHOD AND USE THEREOF

(71) Applicant: Guangdong GreenTech Lemon Innovations Co., Ltd., Qingyuan (CN)

(72) Inventor: Jinsong LIAO, Guangzhou (CN)

(73) Assignee: Guangdong GreenTech Lemon Innovations Co., Ltd., Qingyuan (CN)

(21) Appl. No.: 18/640,008

(22) Filed: Apr. 19, 2024

(30) Foreign Application Priority Data
Apr. 21, 2023 (CN) 202310440658.3

(51) Int. Cl. A61K 36/73; A61K 36/28; A61K 36/31; A61P 11/00

(52) U.S. Cl. CPC A61K 36/73; A61K 36/28; A61K 36/31; A61P 11/00; A61K 2236/15; A61K 2236/333; A61K 2236/53

(57) ABSTRACT

A plant extract for alleviating pulmonary fibrosis, and a preparation method and a use thereof are provided. The preparation method includes the following steps: (1) crushing Aronia melanocarpa (Michx.) Elliott berries after drying, mixing the crushed berries with ethanol for soaking and extraction, and filtering the mixture to obtain an extract solution of Aronia melanocarpa (Michx.) Elliott; (2) crushing Cynara scolymus L. buds after drying, mixing the crushed buds with ethanol for extraction, and filtering the mixture to obtain an extract solution of Cynara scolymus L.; (3) crushing Brassica oleracea var. albiflora Kuntze roots after drying, and mixing the crushed roots with ethanol for ultrasonic extraction to obtain an extract solution of Brassica oleracea var. albiflora Kuntze; (4) mixing the extract solutions of Aronia melanocarpa (Michx.) Elliott, Cynara scolymus L. and Brassica oleracea var. albiflora Kuntze to obtain the plant extract for alleviating pulmonary fibrosis.

### CROSS REFERENCE TO THE RELATED APPLICATIONS

This application is based upon and claims priority to Chinese Patent Application No. 202310440658.3, filed on Apr. 21, 2023, the entire contents of which are incorporated herein by reference.

### TECHNICAL FIELD

The present invention relates to the technical field of plant extraction, and in particular to a plant extract for alleviating pulmonary fibrosis, and a preparation method and a use thereof.

### BACKGROUND

Pulmonary fibrosis is an end-stage change of a large group of lung diseases characterized by the proliferation of fibroblasts and the aggregation of a large amount of extracellular matrixes, which are accompanied with inflammatory damage and tissue structure destruction, and has the main manifestations of dry cough, progressive dyspnea, and more obvious symptoms after exercise. Patients in the end stage may have hypoxia manifestations such as dizziness, purple lips and nails, and limited movement. In recent years, the morbidity and mortality of pulmonary fibrosis have been increasing year by year. The main measures to treat pulmonary fibrosis include glucocorticoids, immunosuppressants and cytotoxic medicaments. However, long-term use of the above medicaments has many side effects along with an unobvious therapeutic effect. The clinical research results show that there is no significant change in the survival rate of patients with pulmonary fibrosis treated with glucocorticoids. Therefore, an urgent problem to be solved currently is to find a medicament for effectively alleviating pulmonary fibrosis.

Aronia melanocarpa (Michx.) Elliott is a deciduous bushy shrub of the Aronia genus, the Rosaceae family, with its berries rich in flavonoids, anthocyanins, polyphenols and other substances, and its extracts effective for the treatment of cancers, diabetes, cardiovascular and cerebrovascular diseases and other diseases. Cynarascolymus L. is a perennial herbaceous plant of the Cynara L. genus, the Asteraceae family and contains substances such as cardium, asparagine, and flavonoid compounds. Regular consumption has the effects of protecting the liver and kidneys, enhancing liver detoxification, promoting digestion, improving blood circulation, preventing arteriosclerosis, and protecting cardiovascular system. Brassica oleracea var. albiflora Kuntze is an annual herbaceous plant of the Brassica L. genus, the Brassicaceae family, with its roots, stems and leaves capable of being used as medicines, and has the effects of detoxifying and relieving sore-throat, smoothing the qi and reducing phlegm, relieving asthma, lowering cholesterol, softening blood vessels, and preventing heart diseases, etc. However, so far, there have been no relevant reports that the above plant extracts can alleviate pulmonary fibrosis.

### SUMMARY

The object of the present invention is to provide a plant extract for alleviating pulmonary fibrosis, and a preparation method and a use thereof. The plant extract obtained by the preparation method of the present invention has the effect of alleviating pulmonary fibrosis, and the plant-derived ingredients used have few side effects and significant effects.

In order to achieve the above-mentioned object of the invention, the present invention provides the following technical solutions:
the present invention provides a preparation method of a plant extract for alleviating pulmonary fibrosis, comprising the following steps:
(1) crushing Aronia melanocarpa (Michx.) Elliott berries after drying, mixing the crushed berries with ethanol for soaking and extraction, and filtering the mixture to obtain an extract solution of Aronia melanocarpa (Michx.) Elliott;
(2) crushing Cynara scolymus L. buds after drying, mixing the crushed buds with ethanol for extraction, and filtering the mixture to obtain an extract solution of Cynara scolymus L.;
(3) crushing Brassica oleracea var. albiflora Kuntze roots after drying, and mixing the crushed roots with ethanol for ultrasonic extraction to obtain an extract solution of Brassica oleracea var. albiflora Kuntze; and
(4) mixing the extract solutions of Aronia melanocarpa (Michx.) Elliott, Cynara scolymus L. and Brassica oleracea var. albiflora Kuntze to obtain the plant extract for alleviating pulmonary fibrosis.

Preferably, the ratio of the dried Aronia melanocarpa (Michx.) Elliott berries to the ethanol in the step (1) is 1 g: 5-10 mL, and the percentage concentration by volume of the ethanol is 70-80%.

Preferably, the soaking in the step (1) is for a period of 10-20 h; and

the extracting is under a temperature of 50-60° C. and is conducted 2-4 times, each for a period of 1-3 h.

Preferably, the ratio of the dried Cynara scolymus L. buds to the ethanol in the step (2) is 1 g: 8-15 mL, and a volume percentage concentration of the ethanol is 60-70%.

Preferably, the extracting in the step (2) is under a temperature of 50-60° C., and is conducted is 1-2 times, each for a period of 2-3 h.

Preferably, the ratio of the dried Brassica oleracea var. albiflora Kuntze roots to the ethanol in the step (3) is 1 g: 5-10 mL, and the percentage concentration by volume of the ethanol is 60-80%.

Preferably, the ultrasonic extraction in the step (3) is under a temperature of 40-50° C., for a period of 0.5-2 h, and at a power of 50-100 W and a frequency of 40-60 KHz.

Preferably, the concentrations of the extract solutions of Aronia melanocarpa (Michx.) Elliott, Cynara scolymus L. and Brassica oleracea var. albiflora Kuntze in the step (4) are on the basis of preparing 1 mL of the extract solution from 0.5-1 g of dry raw materials; and the volume ratio of the extract solutions of Aronia melanocarpa (Michx.) Elliott, Cynara scolymus L. and Brassica oleracea var. albiflora Kuntze is 1:(1-3):(1-2).

The present invention further provides a plant extract solution prepared by the above-mentioned preparation method.

The present invention further provides a use of the above-mentioned plant extract solution in preparing a product for alleviating pulmonary fibrosis.

Compared with the prior art, the present invention has the following beneficial effects:

1. The preparation method of the plant extract of the present invention is simple, and the prepared plant extract can significantly increase the body weight in rat models of pulmonary fibrosis, and significantly reduce the lung coefficient and the hydroxyproline content in lung tissues in rat models, thus indicating that the plant extract of the present invention shows a good effect of alleviating pulmonary fibrosis.

2. The Aronia melanocarpa (Michx.) Elliott, Cynara scolymus L. and Brassica oleracea var. albiflora Kuntze used in the present invention have wide sources, low costs and wide application values.

### BRIEF DESCRIPTION OF THE DRAWINGS

FIG. 1 shows the influence of a plant extract of the present invention on the body weight in rat models;

FIG. 2 shows the influence of a plant extract of the present invention on the lung coefficient in rat models; and

FIG. 3 shows the influence of a plant extract of the present invention on the hydroxyproline content in lung tissues in rat models.

### DETAILED DESCRIPTION OF THE EMBODIMENTS

the present invention provides a preparation method of a plant extract for alleviating pulmonary fibrosis, comprising the following steps:

(1) crushing Aronia melanocarpa (Michx.) Elliott berries after drying, mixing the crushed berries with ethanol for soaking and extraction, and filtering the mixture to obtain an extract solution of Aronia melanocarpa (Michx.) Elliott;

(2) crushing Cynara scolymus L. buds after drying, mixing the crushed buds with ethanol for extraction, and filtering the mixture to obtain an extract solution of Cynara scolymus L.;

(3) crushing Brassica oleracea var. albiflora Kuntze roots after drying, and mixing the crushed roots with ethanol for ultrasonic extraction to obtain an extract solution of Brassica oleracea var. albiflora Kuntze; and

(4) mixing the extract solutions of Aronia melanocarpa (Michx.) Elliott, Cynara scolymus L. and Brassica oleracea var. albiflora Kuntze to obtain the plant extract for alleviating pulmonary fibrosis.

In the present invention, the Aronia melanocarpa (Michx.) Elliott berries are crushed after drying, mixed with ethanol for soaking and extraction, and filtered to obtain an extract solution of Aronia melanocarpa (Michx.) Elliott, and the method for drying and crushing the Aronia melanocarpa (Michx.) Elliott berries is a conventional method in the art.

In the present invention, the ratio of the dried Aronia melanocarpa (Michx.) Elliott berries to the ethanol is 1 g: 5-10 mL, preferably 1 g: 6-9 mL, further preferably 1 g: 7-8 mL; and the percentage concentration by volume of the ethanol is 70-80%, preferably 72-78%, further preferably 74-76%.

In the present invention, the soaking is for a period of 10-20 h, preferably 12-18 h, further preferably 14-16 h.

In the present invention, the extracting is under a temperature of 50-60° C., preferably 52-58° C., further preferably 54-56° C.; the extracting is conducted 2-4 times, preferably 3 times; and each extracting is for a period of 1-3 h, preferably 1.5-2.5 h, further preferably 2 h.

In the present invention, the filtering is performed by a conventional filtering method in the art.

In the present invention, the Cynara scolymus L. buds crushed after drying, mixed with ethanol for extraction, and filtered to obtain an extract solution of Cynara scolymus L., and the method for drying and crushing the Cynara scolymus L. buds is a conventional method in the art.

In the present invention, the ratio of the dried Cynara scolymus L. buds to the ethanol is 1 g: 8-15 mL, preferably 1 g: 9-14 mL, further preferably 1 g: 10-12 mL; and the percentage concentration by volume of the ethanol is 60-70%, preferably 62-68%, further preferably 64-66%.

In the present invention, the extracting is under a temperature of 50-60° C., preferably 52-58° C., further preferably 54-56° C.; the extracting is conducted 1-2 times; and each extracting is for a period of 2-3 h, preferably 2.5 h.

In the present invention, the filtering is performed by a conventional filtering method in the art.

In the present invention, the Brassica oleracea var. albiflora Kuntze roots are crushed after drying, and mixed with ethanol for ultrasonic extraction to obtain an extract solution of Brassica oleracea var. albiflora Kuntze, and the method for drying and crushing the Brassica oleracea var. albiflora Kuntze roots is a conventional method in the art.

In the present invention, the ratio of the dried Brassica oleracea var. albiflora Kuntze roots to the ethanol is 1 g: 5-10 mL, preferably 1 g: 6-9 mL, further preferably 1 g: 7-8 mL; and the percentage concentration by volume of the ethanol is 60-80%, preferably 65-75%, further preferably 68-72%.

In the present invention, the ultrasonic extraction is under a temperature of 40-50° C., preferably 42-48° C., further preferably 44-46° C.; the ultrasonic extraction is for a period of 0.5-2 h, preferably 1-1.5 h; the ultrasonic extraction is at a power of 50-100 W, preferably 60-90 W, further preferably 70-80 W; and the ultrasonic extraction is at a frequency of 40-60 kHz, preferably 45-55 kHz, further preferably 48-52 kHz.

In the present invention, the extract solutions of Aronia melanocarpa (Michx.) Elliott, Cynara scolymus L. and Brassica oleracea var. albiflora Kuntze are mixed to obtain the plant extract for alleviating pulmonary fibrosis. The concentrations of the extract solutions of Aronia melanocarpa (Michx.) Elliott, Cynara scolymus L. and Brassica oleracea var. albiflora Kuntze are on the basis of preparing 1 mL of the extract solution from 0.5-1 g of dry raw materials; preferably, preparing 1 mL of the extract solution from 0.6-0.9 g of dry raw materials; further preferably, preparing 1 mL of the extract solution from 0.7-0.8 g of dry raw materials. The volume ratio of the extract solutions of Aronia melanocarpa (Michx.) Elliott, Cynara scolymus L. and Brassica oleracea var. albiflora Kuntze is preferably 1:(1-3):(1-2).

The present invention further provides a plant extract solution prepared by the above-mentioned preparation method.

The present invention further provides a use of the above-mentioned plant extract solution in preparing a product for alleviating pulmonary fibrosis.

The technical solutions provided by the present invention will be described in detail below with reference to the embodiments, but these embodiments shall not be understood as limiting the protection scope of the present invention.

### Embodiment 1

The present embodiment provides a preparation method of a plant extract for alleviating pulmonary fibrosis, comprising the following steps:

(1) crushing Aronia melanocarpa (Michx.) Elliott berries after drying; weighing 100 g of Aronia melanocarpa (Michx.) Elliott berry power and mixing it with 500 mL of ethanol having a volume percentage content of 80%; soaking for 10 h at room temperature, and then heating to 50° C. and extracting for 3 h; extracting the filtered residue with 500 mL of ethanol having a volume percentage content of 80% for 3 h at 50° C.; and combining two filtrates and concentrating the filtrate to the concentration on the basis of preparing 1 mL of the filtrate from 0.5 g of the dried Aronia melanocarpa (Michx.) Elliott berries, to obtain an extract solution of Aronia melanocarpa (Michx.) Elliott.

(2) crushing Cynara scolymus L. buds after drying; weighing 100 g of Cynara scolymus L. bud powder and mixing it with 800 mL of ethanol having a volume percentage content of 70%; heating to 50° C. and extracting for 3 h; then extracting the filtered residue with 800 mL of ethanol having a volume percentage content of 70% for 3 h at 50° C.; and combining two filtrates and concentrating the filtrate to a concentration on the basis of preparing 1 mL of the filtrate from 0.5 g of the dried Cynara scolymus L. buds, to obtain an extract solution of Cynara scolymus L.

(3) crushing Brassica oleracea var. albiflora Kuntze roots after drying; and weighing 100 g of Brassica oleracea var. albiflora Kuntze root powder, mixing it with 500 mL of ethanol for ultrasonic extraction under the ultrasonic extraction parameters of a temperature of 40° C., time of 2 h, a power of 50 W and a frequency of 60 kHz, wherein the concentration of the filtered filtrate is on the basis of preparing 1 mL of the filtrate from 0.5 g of the dried Brassica oleracea var. albiflora Kuntze roots, to obtain an extract solution of Brassica oleracea var. albiflora Kuntze.

(4) mixing the above-mentioned extract solutions of Aronia melanocarpa (Michx.) Elliott, Cynara scolymus L. and Brassica oleracea var. albiflora Kuntze at a volume ratio of 1:3:2 to obtain the plant extract for alleviating pulmonary fibrosis.

### Embodiment 2

The present embodiment provides a preparation method of a plant extract for alleviating pulmonary fibrosis, comprising the following steps:

(1) crushing Aronia melanocarpa (Michx.) Elliott berries after drying; weighing 100 g of Aronia melanocarpa (Michx.) Elliott berry powder and mixing it with 1000 mL of ethanol having a volume percentage content of 70%; soaking for 20 h at room temperature, and then heating to 60° C. and extracting for 1 h; extracting the filtered residue twice; and combining three filtrates and concentrating the filtrate to the concentration on the basis of preparing 1 mL of the filtrate from 0.8 g of the dried Aronia melanocarpa (Michx.) Elliott berries, to obtain an extract solution of Aronia melanocarpa (Michx.) Elliott.

(2) crushing Cynara scolymus L. buds after drying; weighing 100 g of Cynara scolymus L. bud powder and mixing it with 1,500 mL of ethanol having a volume percentage content of 60%; heating to 60° C. and extracting for 2 h; then extracting the filtered residue with 1,500 mL of ethanol having a volume percentage content of 60% for 2 h at 60° C.; and combining two filtrates and concentrating the filtrate to a concentration on the basis of preparing 1 mL of the filtrate from 0.8 g of the dried Cynara scolymus L. buds, to obtain an extract solution of Cynara scolymus L.

(3) crushing Brassica oleracea var. albiflora Kuntze roots after drying; and weighing 100 g of Brassica oleracea var. albiflora Kuntze root powder, mixing it with 1,000 mL of ethanol for ultrasonic extraction under the ultrasonic extraction parameters of a temperature of 50° C., time of 0.5 h, a power of 100 W and a frequency of 40 kHz, wherein the concentration of the filtered filtrate is on the basis of preparing 1 mL of the filtrate from 0.8 g of the dried Brassica oleracea var. albiflora Kuntze roots, to obtain an extract solution of Brassica oleracea var. albiflora Kuntze.

(4) mixing the above-mentioned extract solutions of Aronia melanocarpa (Michx.) Elliott, Cynara scolymus L. and Brassica oleracea var. albiflora Kuntze at a volume ratio of 1:2:2 to obtain the plant extract for alleviating pulmonary fibrosis.

### Embodiment 3

The present embodiment provides a preparation method of a plant extract for alleviating pulmonary fibrosis, comprising the following steps:

(1) crushing Aronia melanocarpa (Michx.) Elliott berries after drying; weighing 100 g of Aronia melanocarpa (Michx.) Elliott berry power and mixing it with 800 mL of ethanol having a volume percentage content of 75%; soaking for 15 h at room temperature, and then heating to 55° C. and extracting for 2 h; extracting the filtered residue with 800 mL of ethanol having a volume percentage content of 75% for 2 h at 55° C.; and combining two filtrates and concentrating the filtrate to the concentration on the basis of preparing 1 mL of the filtrate from 1 g of the dried Aronia melanocarpa (Michx.) Elliott berries, to obtain an extract solution of Aronia melanocarpa (Michx.) Elliott.

(2) crushing Cynara scolymus L. buds after drying; weighing 100 g of Cynara scolymus L. bud powder and mixing it with 1,000 mL of ethanol having a volume percentage content of 65%; heating to 55° C. and extracting for 2.5 h; then extracting the filtered residue with 1,000 mL of ethanol having a volume percentage content of 65% for 2.5 h at 55° C.; and combining two filtrates and concentrating the filtrate to a concentration on the basis of preparing 1 mL of the filtrate from 1 g of the dried Cynara scolymus L. buds, to obtain an extract solution of Cynara scolymus L.

(3) crushing Brassica oleracea var. albiflora Kuntze roots after drying; and weighing 100 g of Brassica oleracea var. albiflora Kuntze root powder, mixing it with 800 mL of ethanol for ultrasonic extraction under the ultrasonic extraction parameters of a temperature of 45° C., time of 1 h, a power of 80 W and a frequency of 50 kHz, wherein the concentration of the filtered filtrate is on the basis of preparing 1 mL of the filtrate from 1 g of the dried Brassica oleracea var. albiflora Kuntze roots, to obtain an extract solution of Brassica oleracea var. albiflora Kuntze.

(4) mixing the above-mentioned extract solutions of Aronia melanocarpa (Michx.) Elliott, Cynara scolymus L. and Brassica oleracea var. albiflora Kuntze at a volume ratio of 1:2:1 to obtain the plant extract for alleviating pulmonary fibrosis.

### Comparative Example 1

Compared with Embodiment 1, the present comparative example does not contain an extract solution of Brassica oleracea var. albiflora Kuntze, and the remaining preparation methods and parameters are the same as those in Embodiment 1.

### Comparative Example 2

Compared with Embodiment 1, the volume ratio of the extract solutions of Aronia melanocarpa (Michx.) Elliott, Cynara scolymus L. and Brassica oleracea var. albiflora Kuntze in the present comparative example is 1:1:1, and the remaining preparation methods and parameters are the same as those in Embodiment 1.

### Experimental Example 1

The present experimental example explored the influence of the plant extract solution prepared in Embodiments 1-3 on the body weight and lung coefficient of rats. The method was as follows:

### 1. Construction and Grouping of Rat Models of Pulmonary Fibrosis

80 healthy SD rats including half males and half females with a body weight of 230 g±10 g were selected and divided into a blank group, a model group, embodiment group 1, embodiment group 2, embodiment group 3, comparative example group 1, comparative example group 2 and a positive control group, each group consisting of 10 rats. After the rats in each group were raised regularly for one week, each rat was intraperitoneally injected with 4% chloral hydrate at an injection dose of 10 ml/kg. The anesthetized rats were fixed in a supine position, and the rat necks were subjected to hair removal, disinfected with iodine alcohol, and longitudinally cut to expose tracheas. Except for the blank group, the rats in each group were pierced with an injector by 1 cm toward the heart through a gap between two tracheal cartilage rings, and after resistance-free withdrawal, bleomycin was injected into the trachea at a dose of 5 mg/kg. The blank group was injected with an equal volume of normal saline. Immediately after injection, the rats were rotated upright for 5 min to fully distribute the medicament in the lungs. Various layers of tissues were sutured, and local alcohol disinfection was performed. 24 hours after modeling, the rats in the blank group and the model group were intragastrically administered with normal saline at a dose of 5 mL/kg every day, the rats in the positive control group were intragastrically administered with prednisone acetate at a dose of 5 mL/kg every day, and the rats in embodiment groups 1-3 and comparative example groups 1-2 were intragastrically administered with corresponding plant extract at a dose of 5 mL/kg continuously for 28 d. During this period, the rats in each group were raised according to a conventional method.

### 2. Detection Indicators

(1) Body weight: weighed the rats in each group on the day of intragastric administration, and weighed the rats in each group every 7 d.

(2) Lung coefficient: 5 rats in each group were killed on the 14th and 28th days, and the lung tissues were removed and weighed to calculate the lung coefficient: lung coefficient=wet lung weight (mg)/body weight (mg)×100%.

### 3. Experimental Results

### (1) Body Weight Measurement Results

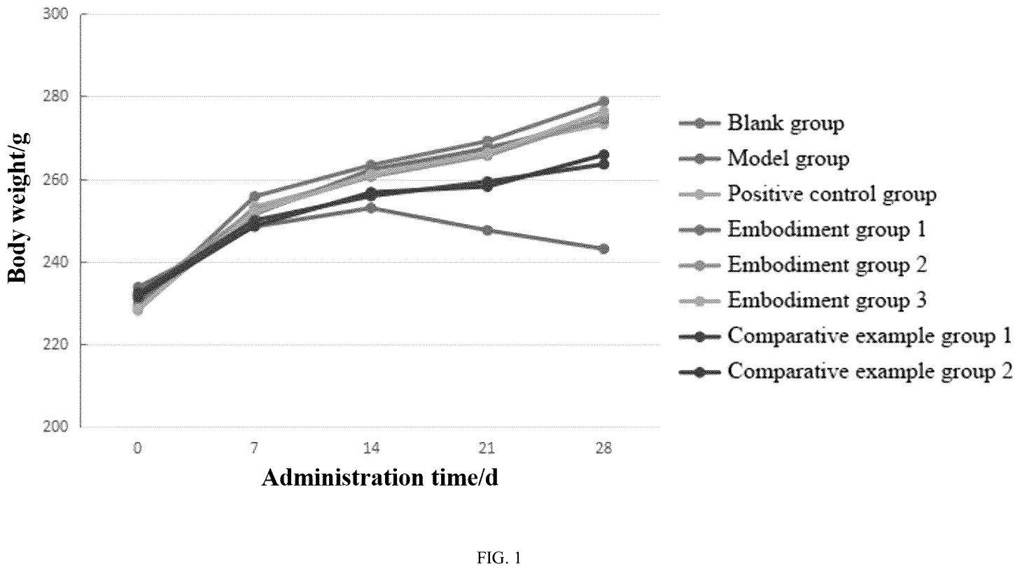

The results are shown in Table 1 and FIG. 1. It can be seen from Table 1 and FIG. 1 that compared with the body weight of the rats in the blank group, the body weight of the rats in the model group decreased significantly and the difference was statistically significant (P<0.05). Compared with the body weight of the rats in the model group, the body weight of rats in embodiment groups 1-3 increased significantly (P<0.05), indicating that the plant extracts prepared in Embodiments 1-3 can improve the physical fitness of rat models of pulmonary fibrosis and significantly alleviate the influence of pulmonary fibrosis on rats.

TABLE 1
Changes in body weight of rats in each group

| | Initial | Weight on the | Weight on the | Weight on the | Weight on the |
|---|---|---|---|---|---|
| Group | weight/g | 7th day/g | 14th day/g | 21st day/g | 28th day/g |
| Blank group | 229.2 | 255.7 | 263.3 | 269.1 | 278.7 |
| Model group | 233.7 | 248.4 | 252.9 | 247.5 | 243.0 |
| Positive control | 228.1 | 253.2 | 260.7 | 267.6 | 273.2 |
| group | | | | | |
| Embodiment | 230.6 | 251.4 | 262.2 | 267.3 | 274.5 |
| group 1 | | | | | |
| Embodiment | 231.9 | 252.3 | 260.5 | 265.6 | 275.0 |
| group 2 | | | | | |
| Embodiment | 228.5 | 251.9 | 261.0 | 266.2 | 276.3 |
| group 3 | | | | | |
| Comparative | 232.3 | 249.9 | 255.8 | 259.3 | 263.5 |
| example group 1 | | | | | |
| Comparative | 231.4 | 248.6 | 256.7 | 258.1 | 265.8 |
| example group 2 | | | | | |

### (2) Lung Coefficient Measurement Results

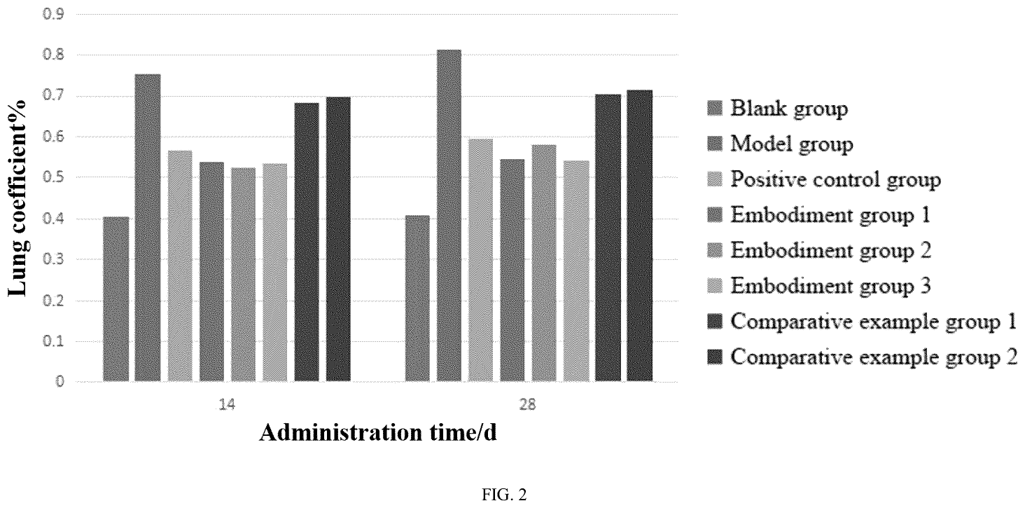

The results are shown in Table 2 and FIG. 2. It can be seen from Table 2 and FIG. 2 that compared with the lung coefficient of the rats in the blank group, the lung coefficient of the rats in the model group increased significantly and the difference was statistically significant (P<0.05). After administration for 14 d and 28 d, compared with the lung coefficient of the rats in the model group, the lung coefficient of the rats in embodiment group 1-3 decreased significantly (P<0.05), indicating that the plant extracts prepared in Embodiments 1-3 can improve bleomycin-induced pulmonary fibrosis in mice and significantly alleviate the development of pulmonary fibrosis in mouse models.

TABLE 2
Changes in lung coefficient of rats in each group

| | Lung coefficient | Lung coefficient |
|---|---|---|
| Group | on the 14th day/% | on the 28th day/% |
| Blank group | 0.405 | 0.409 |
| Model group | 0.753 | 0.812 |
| Positive control group | 0.568 | 0.594 |
| Embodiment group 1 | 0.537 | 0.546 |
| Embodiment group 2 | 0.525 | 0.579 |
| Embodiment group 3 | 0.533 | 0.542 |
| Comparative example group 1 | 0.681 | 0.705 |
| Comparative example group 2 | 0.696 | 0.713 |

### Experimental Example 2

The present experimental example explored the influence of the plant extracts prepared in Embodiments 1-3 on the hydroxyproline content in lung tissues. The method was as follows:

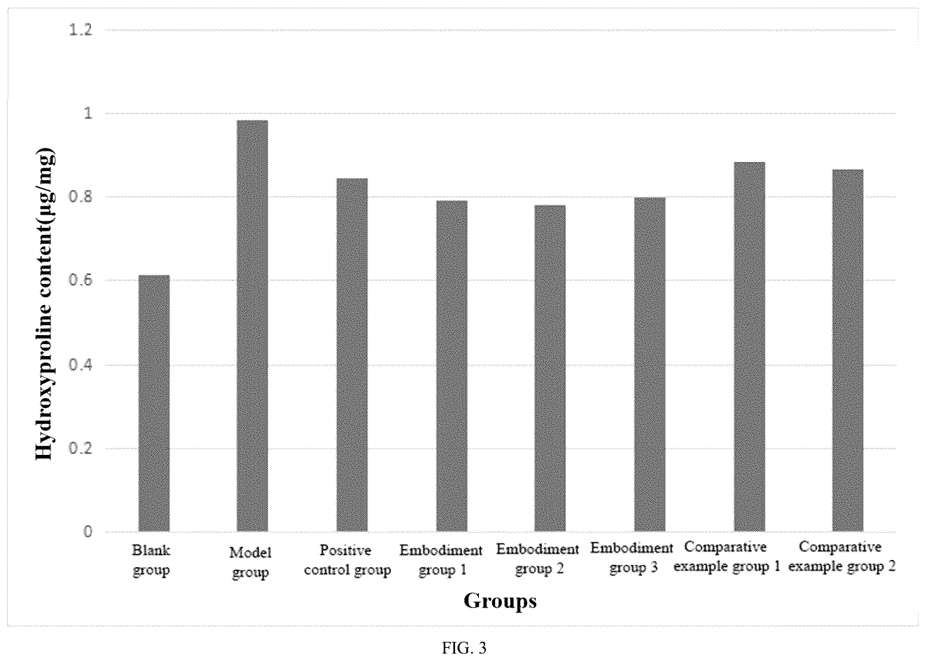

The construction and grouping of the rat pulmonary fibrosis model are as shown in Experimental Example 1. The rats in each group were killed after intragastric administration for 28 d. Hydroxyproline content kit (purchased from Shanghai Enzyme-linked Biotechnology Co., Ltd.) was used to measure the hydroxyproline content in lung tissues. The results are shown in Table 3 and FIG. 3.

It can be seen from Table 3 and FIG. 3 that compared with the hydroxyproline content in lung tissues of the rats in the blank group, the hydroxyproline content in lung tissues of the rats in the model group increased significantly and the difference was statistically significant (P<0.05). Compared with the hydroxyproline content in lung tissues of the rats in the model group, the hydroxyproline content in lung tissues of the rats in embodiment groups 1-3 decreased significantly (P<0.05). The hydroxyproline content in lung tissues is one of the important indicators for judging the degree of lung tissue fibrosis. The above results illustrate that the plant extracts prepared in Embodiments 1-3 can significantly alleviate the development of pulmonary fibrosis in mouse models.

TABLE 3
Comparison of hydroxyproline contents
in lung tissues of rats in each group

| Group | Hydroxyproline content (μg/mg) |
|---|---|
| Blank group | 0.615 |
| Model group | 0.984 |
| Positive control group | 0.847 |
| Embodiment group 1 | 0.793 |
| Embodiment group 2 | 0.782 |
| Embodiment group 3 | 0.801 |
| Comparative example group 1 | 0.885 |
| Comparative example group 2 | 0.867 |

As it can be seen from the above embodiments, the present invention provides a plant extract for alleviating pulmonary fibrosis, and a preparation method and a use thereof. The plant extract obtained by the preparation method of the present invention has the effect of alleviating pulmonary fibrosis, and the plant source ingredients used have few side effects and significant effects.

The above descriptions are only preferred embodiments of the present invention. It shall be noted that those of ordinary skill in the art can further make several improvements and modifications without departing from the principles of the present invention, and these improvements and modifications shall be also regarded as the protection scope of the present invention.

What is claimed is:

1. A preparation method of a plant extract for alleviating pulmonary fibrosis, comprising the following steps:
(1) crushing Aronia melanocarpa (Michx.) Elliott berries after a drying, mixing crushed Aronia melanocarpa (Michx.) Elliott berries with first ethanol for a soaking and a first extraction to obtain a first mixture, and filtering the first mixture to obtain an extract solution of Aronia melanocarpa (Michx.) Elliott;
(2) crushing Cynara scolymus L. buds after the drying, mixing crushed Cynara scolymus L. buds with second ethanol for a second extraction to obtain a second mixture, and filtering the second mixture to obtain an extract solution of Cynara scolymus L.;
(3) crushing Brassica oleracea var. albiflora Kuntze roots after the drying, and mixing crushed Brassica oleracea var. albiflora Kuntze roots with third ethanol for an ultrasonic extraction to obtain an extract solution of Brassica oleracea var. albiflora Kuntze; and
(4) mixing the extract solution of the Aronia melanocarpa (Michx.) Elliott, the extract solution of the Cynara scolymus L., and the extract solution of the Brassica oleracea var. albiflora Kuntze to obtain the plant extract for alleviating the pulmonary fibrosis;
wherein
a ratio of dried Aronia melanocarpa (Michx.) Elliott berries to the first ethanol in the step (1) is 1 g: 5-10 mL, and a percentage concentration by volume of the first ethanol is 70-80%;
the soaking in the step (1) is for a period of 10-20 h;
the first extraction in the step (1) is under a temperature of 50-60° C., and is conducted 2-4 times each for a period of 1-3 h;
a ratio of dried Cynara scolymus L. buds to the second ethanol in the step (2) is 1 g: 8-15 mL, and a percentage concentration by volume of the second ethanol is 60-70%;
the second extraction in the step (2) is under a temperature of 50-60° C., and is conducted 1-2 times each for a period of 2-3 h;
a ratio of dried Brassica oleracea var. albiflora Kuntze roots to the third ethanol in the step (3) is 1 g: 5-10 mL, and a percentage concentration by volume of the third ethanol is 60-80%;
the ultrasonic extraction in the step (3) is under a temperature of 40-50° C., for a period of 0.5-2 h, and at a power of 50-100 W and a frequency of 40-60 kHz;
concentrations of the extract solution of the Aronia melanocarpa (Michx.) Elliott, the extract solution of the Cynara scolymus L., and the extract solution of the Brassica oleracea var. albiflora Kuntze in the step (4) are on a basis of preparing 1 mL of the extract solution from 0.5-1 g of dry raw materials; and a volume ratio of the extract solution of the Aronia melanocarpa (Michx.) Elliott, the extract solution of the Cynara scolymus L., and the extract solution of the Brassica oleracea var. albiflora Kuntze is 1:(1-3):(1-2).

2. A plant extract prepared by the preparation method according to claim 1.

3. A method of using the plant extract according to claim 2 in preparing a product for alleviating the pulmonary fibrosis.
